(19) United States
(12) Plant Patent Application Publication
(10) Pub. No.: US 2007/0061928 P1
(43) Pub. Date: Mar. 15, 2007

(54) Blackcurrant plant named 'Blackadder'

(75) Inventors: Catherine Snelling, Christchurch (NZ); Geoffrey Langford, Christchurch (NZ)

Correspondence Address: GREENLEE WINNER AND SULLIVAN P C, 4875 PEARL EAST CIRCLE, SUITE 200, BOULDER, CO, 80301, US

(73) Assignee: The Horticulture and Food Research Institute of New Zealand Limited, Auckland (NZ)

(21) Appl. No.: 11/510,270

(22) Filed: Aug. 25, 2006

Related U.S. Application Data
(60) Provisional application No. 60/711,902, filed on Aug. 26, 2005.

(51) Int. Cl. A01H 5/00

(52) U.S. Cl. PLT/156

(57) ABSTRACT

A new and distinct variety of blackcurrant named ‘Blackadder’, botanically identified as Ribes nigrum is described. The new variety is distinguished from others by its early season bud burst, flowering and harvest. Its bush has an upright habit suitable for machine harvesting. Yields are high and the fruit has high anthocyanin levels and moderate ascorbic acid levels.

### GENUS AND SPECIES OF PLANT CLAIMED

Ribes nigrum

### VARIETY DENOMINATION

Blackadder

### CROSS-REFERENCE TO RELATED APPLICATION

This application claims the benefit of U.S. provisional application No. 60/711,902 filed 26 Aug. 2005.

### BACKGROUND TO THE INVENTION

The new variety of blackcurrant, Ribes nigrum, was created during the course of a planned plant-breeding program carried out at HortResearch, Lincoln, New Zealand. The new variety was selected from a population of seedlings derived from a controlled cross that was made in 1998, between L20 (unpatented) and L31 (unpatented). Both parents originated from the breeding programme in New Zealand.

Seed from the cross was sown in the field in 1999, at ‘Waipuna’ Farm, Canterbury, New Zealand. The original plant of the new variety was selected during the 2000-2001 summer.

The new variety was asexually reproduced as hardwood cuttings in winter 2001 and planted into a selection plot for further evaluation. The resulting plants propagated true to type, demonstrating that the characteristics of the new variety are stable and are transmitted without change through succeeding generations.

### SUMMARY OF THE INVENTION

The major characteristics that the new variety exhibits are:

(a) A vigorous, upright growth habit

(b) Early bud burst, flowering and maturity, where winter chilling exceeds 1000 hrs below 7° C.

(c) One-year old wood in winter is orange-brown

(d) The vegetative buds in winter are slightly held out and are ovate in shape.

(e) Ability to bear black, round fruit of good quality in high yields, well suited to juice.

(f) Susceptible to gall mite.

### BRIEF DESCRIPTION OF THE DRAWINGS

The following figures show typical specimens of the new variety in colour as true as is reasonably possible.

FIG. 1: Upright growth habit of bush, typical of the blackcurrant variety ‘Blackadder’.

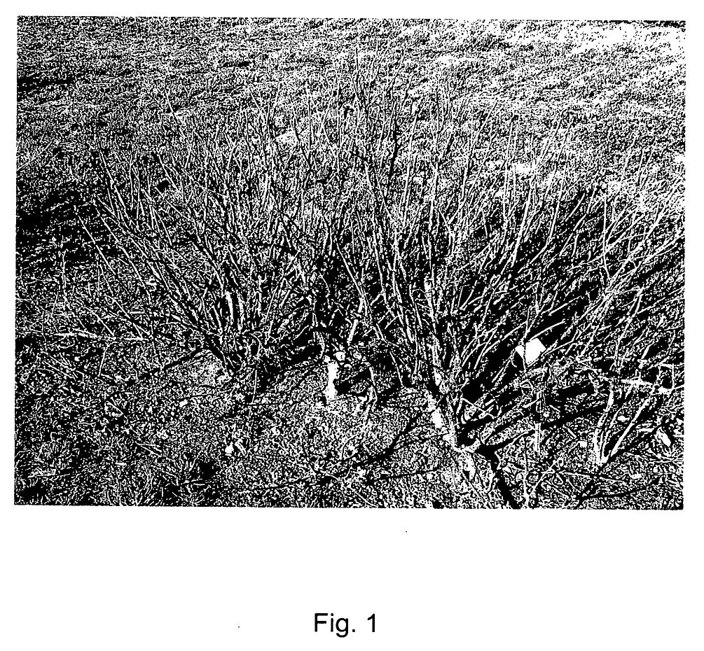

FIG. 2: Colour of wood of one-year old shoots, typical of the blackcurrant variety ‘Blackadder’.

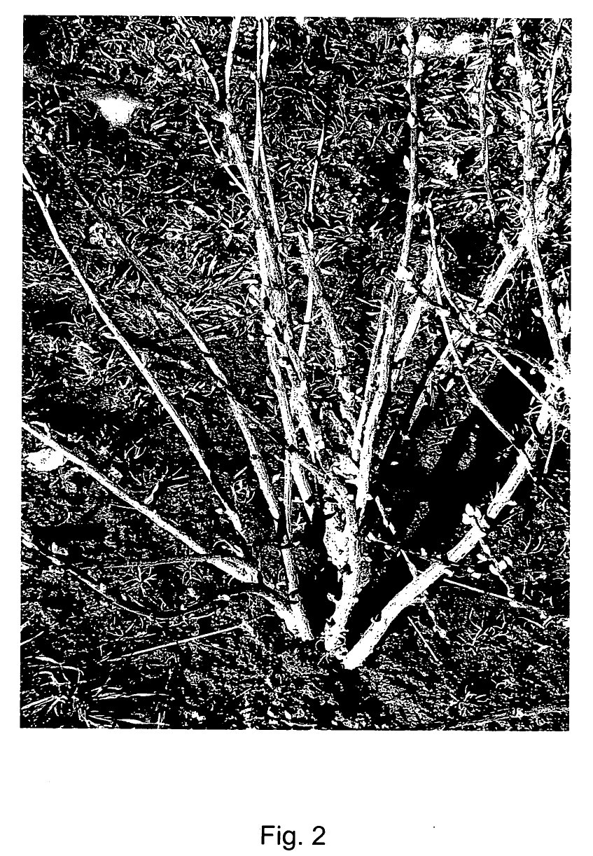

FIG. 3: Typical fruit of blackcurrant variety ‘Blackadder’.

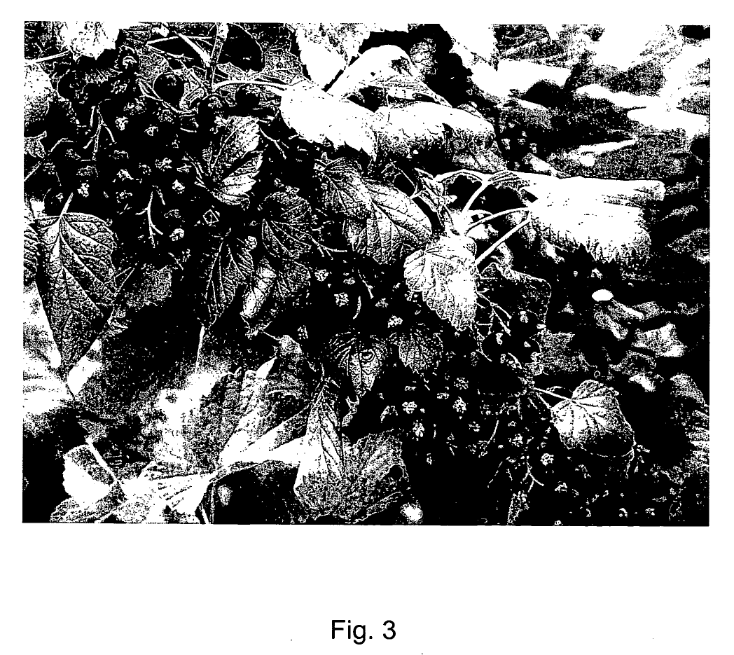

FIG. 4: Typical plant of blackcurrant variety ‘Blackadder’.

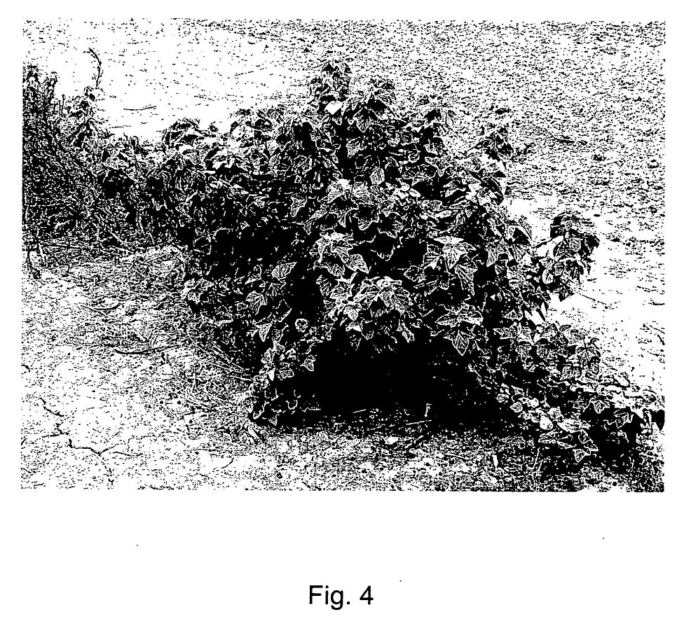

FIG. 5: Close up view of typical fruit of blackcurrant variety ‘Blackadder’.

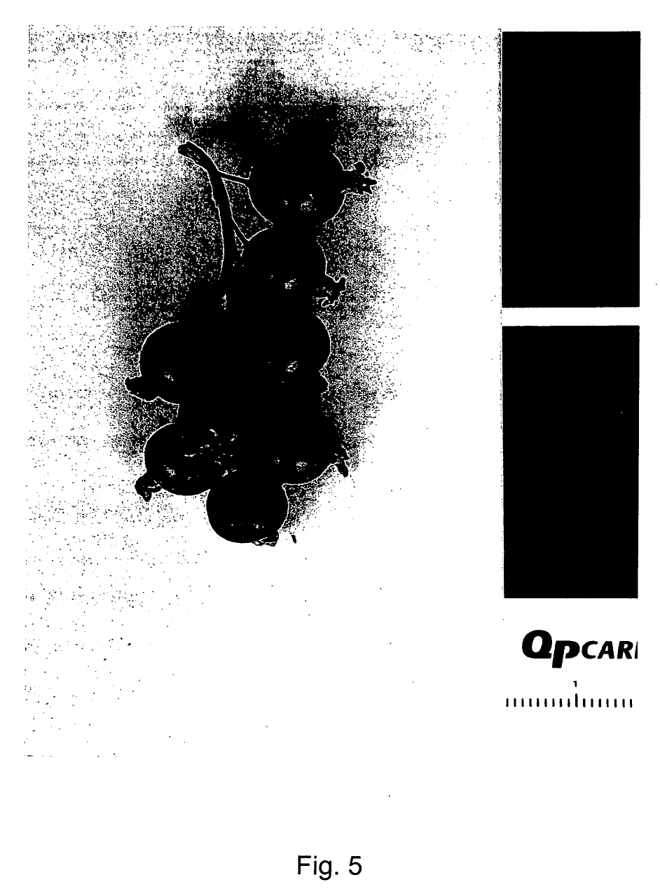

FIG. 6: Vegetative buds in winter, typical of blackcurrant variety ‘Blackadder’.

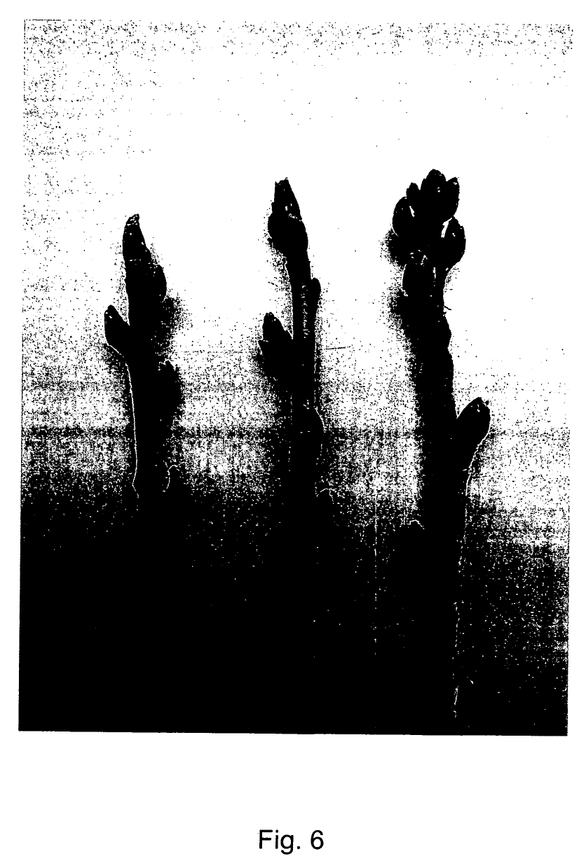

### DETAILED DESCRIPTION OF THE INVENTION

The following is a detailed description of the new variety. The specimens described were grown at ‘Waipuna’ farm, Canterbury, New Zealand. The observations were made in the 2005/2006 season, on plants that were planted in 2001 and managed under standard farm practices.

Horticultural terminology is used in accordance with UPOV guidelines for blackcurrant. All dimensions in millimetres, weights in grams (unless otherwise stated). Colour references refer to the RHS Colour Chart, The Royal Horticultural Society, London. (4^{th} edition, 2001).
Plant and foliage: The plant exhibits an upright growth habit, although over the harvest period the branches tend to branch out with a full crop. Four year old bushes commonly have a height around 1000 mm, although this may vary with growing conditions. The number of basal shoots in unpruned, four year old bushes is typically around 6. One year old wood in winter is an orange-brown colour (greyed orange group 167A). Dormant buds are slightly held out in relation to the shoot. The buds are medium in length around 0.9 cm, ovate in shape and the shape of the bud apex is obtuse. The buds have a medium intensity of anthocyanin colouration and bloom. Young vegetative shoots have a medium intensity of anthocyanin colouration. The first mature leaf typically averages approximately 90 mm in length and approximately 90 mm in width. The form of the base of the leaf is moderately open. The upper surface of the leaf is medium green in colour (green group 137A) with moderate gloss. The leaf petiole has weak anthocyanin colour at the base and distal ends.
Inflorescence: The attitude of the inflorescence is outwards in relation to the shoot. Predominantly the number of inflorescences per bud is usually at least 2. The length of the inflorescence typically averages 50 mm and the number of flowers per inflorescence typically averages 8. The intensity of anthocyanin colouration of the sepal is weak and of the ovary, absent to very weak.
Fruit: The fruit are overall medium in size, averaging 0.8 g. The degree of variability in berry size is moderate, typically ranging from 0.5-1 g. The fruit colour is black (black group 202A) and has medium glossiness. The fruit are round in shape. At maturity the fruit sweetness averages approximately 16° Brix. Ascorbic acid levels are medium, ranging from 125-150 mg/100 g and the total anthocyanin content is typically around 460 mg/100 g. Yields are high averaging approximately 15 tonnes/hectare.
Cultivation: Bud burst is early, late August in New Zealand. Flowering usually commences in September and is early in relation to other cultivars. Fruit is harvested early in the blackcurrant harvest season in New Zealand, in early January. The crop is suitable for machine harvest.
Pest and disease resistance: No pest and disease resistance was observed. Blackadder was found to be susceptible to gall mite (Cecidophyopsis ribis).

We claim:

1. A new and distinct variety of blackcurrant named ‘Blackadder’ is substantially as herein illustrated and described.
